(19) United States
(12) Patent Application Publication
(10) Pub. No.: US 2025/0288361 A1
(43) Pub. Date: Sep. 18, 2025

(54) GENERATING IMAGING POSE RECOMMENDATIONS

(71) Applicant: AURIS HEALTH, INC., Santa Clara, CA (US)

(72) Inventor: Mohammad Matinfar, San Jose, CA (US)

(73) Assignee: AURIS HEALTH, INC., Santa Clara, CA (US)

(21) Appl. No.: 18/861,151

(22) PCT Filed: May 30, 2023
(86) PCT No.: PCT/IB2023/055514

§ 371 Date: Oct. 28, 2024

Related U.S. Application Data
(60) Provisional application No. 63/347,009, filed on May 30, 2022.

(51) Int. Cl. A61B 34/20

(52) U.S. Cl. CPC A61B 34/20; A61B 2034/2051; A61B 2034/2065

(57) ABSTRACT

A method for generating a pose recommendation for an external imaging device may include obtaining location data from one or more location sensors embedded in a medical instrument, the location data relating to locations of the medical instrument over a time period in a coordinate system, determining a medical instrument positioning and a medical instrument tip orientation using the location data, and determining the pose recommendation for the external imaging device using the medical instrument positioning and the medical instrument tip orientation. A system may be configured to perform this method.

### RELATED APPLICATION(S)

This application claims priority to U.S. Provisional Application No. 63/347,009, filed May 30, 2022, entitled GENERATING IMAGING POSE RECOMMENDATIONS, the disclosure of which is hereby incorporated by reference in its entirety.

### BACKGROUND

Various medical procedures involve the use of one or more devices configured to penetrate the human anatomy to reach a treatment site. Certain operational processes can involve localizing a medical instrument within the patient and visualizing an area of interest within the patient. To do so, many medical instruments may include vision capabilities, such as embedded cameras or compatibility with vision probes. External imaging modalities such as a fluoroscopic/x-ray scanning device can provide additional contextual information of a robotic bronchoscope inside a patient's body.

### BRIEF DESCRIPTION OF THE DRAWINGS

Various embodiments are depicted in the accompanying drawings for illustrative purposes and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIG. 1 illustrates an example pose recommendation system for performing various medical procedures in accordance with aspects of the present disclosure.

FIG. 2 is a diagram illustrating components and subsystems of the control system shown in FIG. 1, according to an example embodiment.

FIG. 3 is a flow-chart illustrating a method for generating a pose recommendation for an external imaging device, according to an example embodiment.

FIG. 4 illustrates the distal end of a medical instrument within an anatomical lumen, in accordance with an exemplary embodiment.

FIG. 5 is a diagram illustrating an imaging vector that is based on a medical instrument positioning and a medical instrument orientation, according to an example embodiment.

FIG. 6 is a diagram showing the imaging vector shown in FIG. 5 projected onto the X-Y plane of the patient coordinate system, according to an example embodiment.

FIG. 7 is a diagram illustrating left and right motions based on different values for the angles created from the projected imaging vector, according to an example embodiment.

FIG. 8 is a diagram of a user interface that includes a suggested imaging angle, according to an example embodiment.

### DETAILED DESCRIPTION

The headings provided herein are for convenience only and do not necessarily affect the scope or meaning of disclosure. Although certain exemplary embodiments are disclosed below, the subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Overview

The present disclosure relates to systems, devices, and methods to generate pose recommendations for an external imaging modality based on location data acquired from operation of a medical instrument. It is to be appreciated that, as used herein, a “pose recommendation” may refer to any individual aspect of a pose or combinations thereof. Thus, a pose recommendation may include a location, an orientation (e.g., an angle), or a combination thereof.

Many medical procedures rely on accurate representations of a patient's anatomy to navigate and control medical instruments within that anatomy. For example, in bronchoscopy, accurate and safe biopsy may depend on accurate alignment between a steerable bronchoscope and a biopsy site, such as a nodule or lesion. Robotic bronchoscopy can include a navigation system to facilitate navigation of the bronchoscope to the biopsy site and provide information helpful in aligning the tip of the bronchoscope with the biopsy site. The navigation system may include a three-dimensional model of the anatomy. In the case of bronchoscopy, the three-dimensional model may include data regarding the structure of the luminal network formed by the airways of the lung. This three-dimensional model can be generated from a preoperative computerized tomography (CT) scan of the patient. During the procedure, the coordinate system of the three-dimensional model is registered with the coordinate system of a location sensor incorporated in the bronchoscope so that the navigation system can provide an estimate of the bronchoscope's location within the luminal network of the lungs. Examples of location sensors include robotized sensors, inertial measurement units (IMUs), fiber-optic shape sensors, electromagnetic (EM) sensors, and camera sensors.

Location sensors have their limitation when being used to provide the navigation functionality. For example, the accuracy of robotized sensors will suffer from their miniaturized sizes, the accuracy of IMUs will suffer from accumulated errors, the accuracy of fiber-optic shape sensors will be affected by environmental temperature, the accuracy of EM sensors will suffer from ferro-magnetic objects and the localization accuracy of camera sensors will suffer from bad quality images. In this regard, interventional imaging modalities (referred to herein as “external imaging devices”) such as a fluoroscopic/x-ray scanning device can provide additional localization information of a robotic bronchoscope inside patient body.

However, if an external imaging device, like a fluoroscopic/x-ray scanning device, is not properly aligned with an area of interest, the resulting fluoroscopic images may not be useful to identify and locate the target. For example, if a C-Arm is looking at the face of scope, the physician may have difficulty discerning how much a biopsy tool has been inserted. Embodiments discussed herein may provide a solution for aligning an external imaging device with an area of interest by generating a pose recommendation for an external imaging device. Images acquired by the external imaging device at the recommended pose can facilitate a comparatively improved image based on a visualization plane that contains a bird's eye view of the medical instrument in relationship to an area of interest.

### Pose Recommendation System

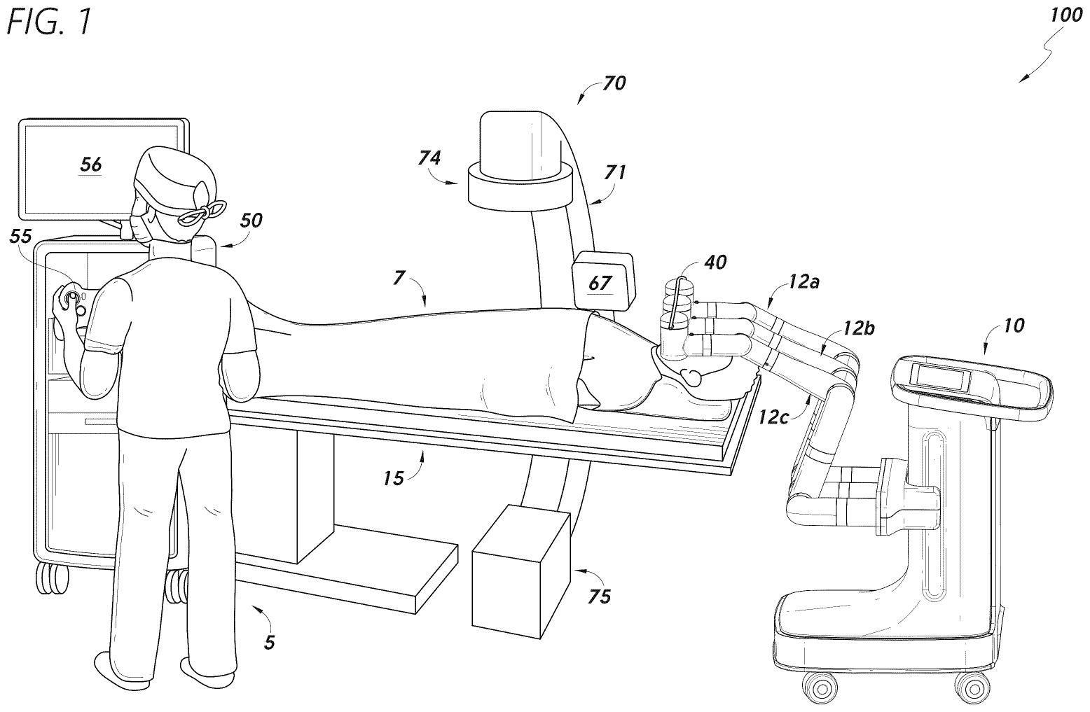

FIG. 1 illustrates an example pose recommendation system 100 for performing various medical procedures in accordance with aspects of the present disclosure. The pose recommendation system 100 may be used for, for example, endoscopic procedures. Robotic medical solutions can provide relatively higher precision, superior control, and/or superior hand-eye coordination with respect to certain instruments compared to strictly manual procedures. Although the system 100 of FIG. 1 is presented in the context of a bronchoscopy procedure, it should be understood that the principles disclosed herein may be implemented in any type of endoscopic procedure.

The pose recommendation system 100 includes a robotic system 10 (e.g., mobile robotic cart) configured to engage with and/or control a medical instrument 40 (e.g., bronchoscope) including a proximal handle and a shaft coupled to the handle at a proximal portion thereof to perform a procedure on a patient 7. It should be understood that the medical instrument 40 may be any type of shaft-based medical instrument, including an endoscope (such as a ureteroscope or bronchoscope), catheter (such as a steerable or non-steerable catheter), needle, nephroscope, laparoscope, or other type of medical instrument. The medical instrument 40 may access the internal patient anatomy through direct access (e.g., through a natural orifice) and/or through percutaneous access via skin/tissue puncture.

The pose recommendation system 100 includes a control system 50 configured to interface with the robotic system 10, provide information regarding the procedure, and/or perform a variety of other operations. For example, the control system 50 can include one or more display(s) 56 configured to present certain information to assist the physician 5 and/or other technician(s) or individual(s). The pose recommendation system 100 can include a table 15 configured to hold the patient 7. The system 100 may further include an electromagnetic (EM) field generator, such as a robot-mounted EM field generator 80 or and EM field generator 85 mounted to the table 15 or other structure.

Although the various robotic arms 12 are shown in various positions and coupled to various tools/devices, it should be understood that such configurations are shown for convenience and illustration purposes, and such robotic arms may have different configurations over time and/or at different points during a medical procedure. Furthermore, the robotic arms 12 may be coupled to different devices/instruments than shown in FIG. 1, and in some cases or periods of time, one or more of the arms may not be utilized or coupled to a medical instrument. Instrument coupling to the robotic system 10 may be via robotic end effectors associated with distal ends of the respective arms 12. The term “end effector” is used herein according to its broad and ordinary meaning and may refer to any type of robotic manipulator device, component, and/or assembly. The terms “robotic manipulator” and “robotic manipulator assembly” are used according to their broad and ordinary meanings and may refer to a robotic end effector and/or sterile adapter or other adapter component coupled to the end effector, either collectively or individually. For example, “robotic manipulator” or “robotic manipulator assembly” may refer to an instrument device manipulator (IDM) including one or more drive outputs, whether embodied in a robotic end effector, adapter, and/or other component(s).

In some embodiments, the physician 5 can interact with the control system 50 and/or the robotic system 10 to cause/control the robotic system 10 to advance and navigate the medical instrument shaft 40 (e.g., a scope) through the patient anatomy to the target site and/or perform certain operations using the relevant instrumentation. The control system 50 can provide information via the display(s) 56 that is associated with the medical instrument 40, such as real-time endoscopic images captured therewith, and/or other instruments of the system 100, to assist the physician 5 in navigating/controlling such instrumentation. The control system 50 may provide imaging/positional information to the physician 5 that is based on certain positioning modalities, such as fluoroscopy, ultrasound, optical/camera imaging, EM field positioning, or other modality, as described in detail herein.

The various scope/shaft-type instruments disclosed herein, such as the medical instrument 40 of the system 100, can be configured to navigate within the human anatomy, such as within a natural orifice or lumen of the human anatomy. The terms “scope” and “endoscope” are used herein according to their broad and ordinary meanings and may refer to any type of elongate (e.g., shaft-type) medical instrument having image generating, viewing, and/or capturing functionality and being configured to be introduced into any type of organ, cavity, lumen, chamber, or space of a body. A scope can include, for example, a ureteroscope (e.g., for accessing the urinary tract), a laparoscope, a nephoscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise an at least partially rigid and/or flexible tube, and may be dimensioned to be passed within an outer sheath, catheter, introducer, or other lumen-type device, or may be used without such devices. Endoscopes and other instruments described herein can have associated with distal ends or other portions thereof certain markers/sensors configured to be visible/detectable in a field/space associated with one or more positioning (e.g., imaging) systems/modalities.

The system 100 is illustrated as including an external imaging device (e.g., a fluoroscopy system) 70, which includes an X-ray generator 75 and an image detector 74 (referred to as an “image intensifier” in some contexts; either component 74, 75 may be referred to as a “source” herein), which may both be mounted on a moveable C-arm 71. The control system 50 or other system/device may be used to store and/or manipulate images generated using the external imaging device 70. In some embodiments, the bed 15 is radiolucent, such that radiation from the generator 75 may pass through the bed 15 and the target area of the patient's anatomy, wherein the patient 7 is positioned between the ends of the C-arm 71. The structure/arm 71 of the fluoroscopy system 70 may be rotatable or fixed. The external imaging device 70 may be implemented to allow live images to be viewed to facilitate image-guided surgery. The structure/arm 71 can be selectively moveable to permit various images of the patient 7 and/or surgical field to be taken by the fluoroscopy panel source 74.

In the example bronchoscopy configuration shown in FIG. 1, the field generator 67 is mounted to the bed. In other example embodiments, the field generator 67 may be mounted to a robotic arm. As the electric field generated by the electric field generator 67 can be distorted by the presence of metal or other conductive components therein, it may be desirable to position the electric field generator 67 in a manner such that other components of the system do not interfere substantially with the electric field. For example, it may be desirable to position the electric field generator 67 at least 8″ or more away from the support arm 71 associated with the fluoroscopy system.

The system 100 (as with other systems disclosed herein) can include an optical imaging source (not shown), such as a camera device (e.g., stereoscopic camera assembly). The optical imaging source may be configured/used to view a field in the surgical environment to identify certain marker(s) disposed in the visual field. For example, in some embodiments, the imaging source may emit infrared (IR) or other-frequency electromagnetic radiation and/or detect reflection of such radiation to identify markers that include surfaces that reflect such radiation. Such optical deflection can indicate position and/or orientation of the marker(s) associated with the particular optical modality. The system 100 can have certain markers/fiducials which may be detectable/positionable in one or more reference/coordinate frames/spaces associated with respective positioning modalities.

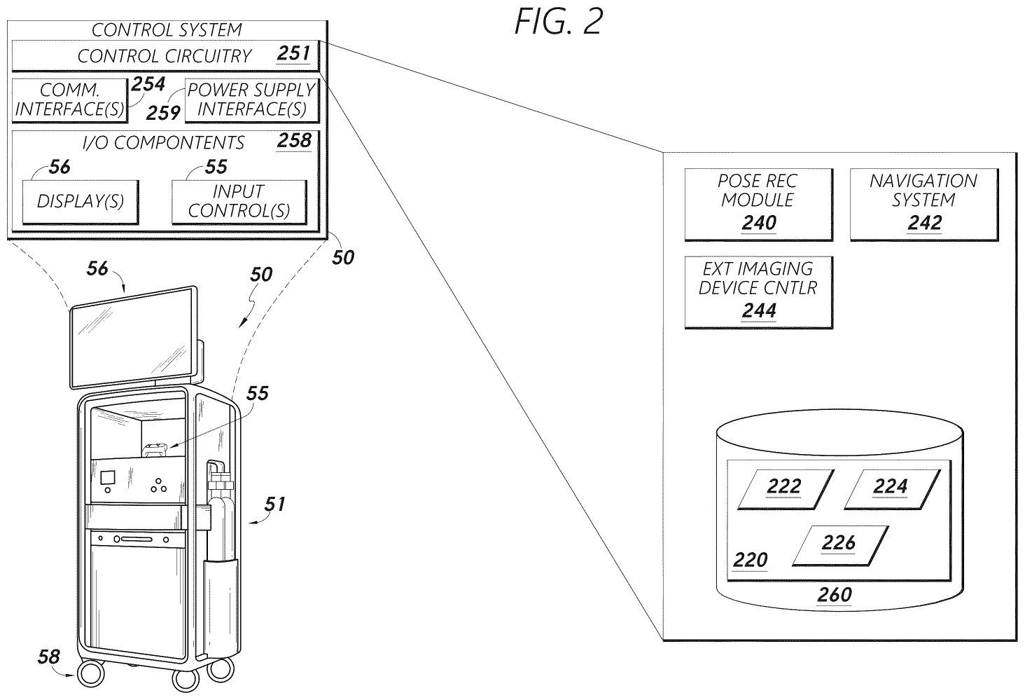

FIG. 2 is a diagram illustrating components and subsystems of the control system 50 shown in FIG. 1, according to an example embodiment. As discussed above, the control system 50 can be configured to provide various functionality to assist in performing a medical procedure. The control system 50 can communicate with the robotic system 10 via a wireless or wired connection (e.g., to control the robotic system 10). In some embodiments, the control system 50 can communicate with the robotic system 10 to receive position data therefrom relating to the position of the distal end of the scope 40 or other instrumentation. Such positioning data may be derived using one or more sensors (e.g., electromagnetic sensors, shape sensing fibers, accelerometers, gyroscopes, satellite-based positioning sensors (e.g., a global positioning system (GPS)), radio-frequency transceivers, and so on) associated with the respective instrumentation and/or based at least in part on robotic system data (e.g., arm position/pose data, known parameters or dimensions of the various system components, etc.) and vision-based algorithms. In some embodiments, the control system 50 can communicate with the EM field generator to control generation of an EM field in an area around the patient 7 and/or around the tracked instrumentation.

As referenced above, the system 100 can include certain control circuitry configured to perform certain of the functionality described herein, including the control circuitry 251 of the control system 50. That is, the control circuitry of the systems 100 may be part of the robotic system 10, the control system 50, or some combination thereof. Therefore, any reference herein to control circuitry may refer to circuitry embodied in a robotic system, a control system, or any other component of a pose recommendation system, such as the pose recommendation systems 100 shown in FIG. 1, respectively. The term “control circuitry” is used herein according to its broad and ordinary meaning, and may refer to any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 251 may be entirely locally maintained/disposed or may be remotely located at least in part (e.g., communicatively coupled indirectly via a local area network and/or a wide area network). Any of the control circuitry 251 may be configured to perform any aspect(s) of the various processes disclosed herein.

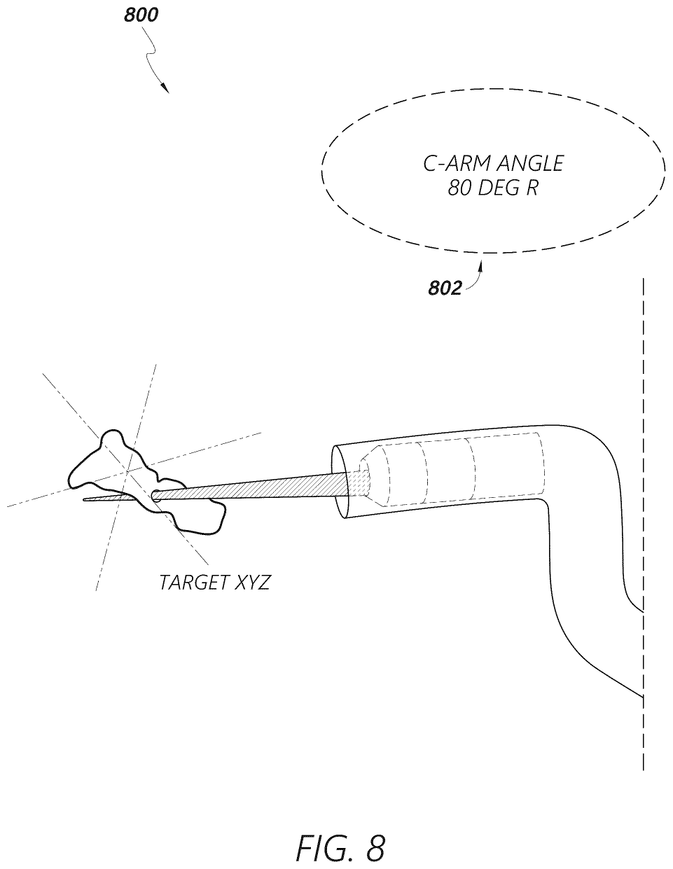

With further reference to FIG. 2, the control system 50 can include various I/O components 258 configured to assist the physician 5 or others in performing a medical procedure. For example, the input/output (I/O) components 258 can be configured to allow for user input to control/navigate the scope 40 and/or instruments within the patient 7. In some embodiments, for example, the physician 5 can provide input to the control system 50 and/or robotic system 10, wherein in response to such input, control signals can be sent to the robotic system 10 to manipulate the scope 40 and/or other robotically-controlled instrumentation. As is discussed below, with reference to FIG. 8, for example, the I/O components 258 may include circuitry from causing the display 56 to render a user interface that displays a recommended imaging angle, as may be computed according to the methods described in this disclosure.

The control system 50 and/or robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore. Such user controls are communicatively and/or physically coupled to respective control circuitry. The control system can include a structural tower 51, as well as one or more wheels 58 that support the tower 51. The control system 50 can further include certain communication interface(s) 254 and/or power supply interface(s) 259.

The control circuitry 251 may include a data store 260 that stores various types of data, such as localization data 220. The localization data 220 may be data representing a location of the scope and may be derived from data generated from the navigation system 242. The localization data 220 may include raw data gathered from and/or processed by input devices (e.g., control system 50, optical sensor, EM sensor, IDM) for generating estimated state information for the instrument as well as output from the navigation system 242. By way of example and not limitation, the localization data 220 may include image data 222, location sensor data 224, and robot data 226. Image data 222 may include one or more image frames captured by the medical instrument (e.g., an imaging device at the distal end of an endoscope), as well as information such as frame rates or timestamps that allow a determination of the time elapsed between pairs of frames. Robot data 226 includes data related to physical movement of the medical instrument or part of the medical instrument (e.g., the instrument tip or sheath) within the tubular network. Example robot data includes command data instructing the instrument tip to reach a specific anatomical site and/or change its orientation (e.g., with a specific pitch, roll, yaw, insertion, and retraction for one or both of a leader and a sheath) within the tubular network, insertion data representing insertion movement of the part of the medical instrument (e.g., the instrument tip or sheath), IDM data, and mechanical data representing mechanical movement of an elongate member of the medical instrument, for example motion of one or more pull-wires, tendons or shafts of the endoscope that drive the actual movement of the medial instrument within the tubular network. Location sensor data 224 may include data collected by location sensors of the instruments, such as EM sensors, shape sensing fibers, and the like. The location sensor data may include data characterizing a pose of the medical instrument in a number of degrees of freedom, such as three degrees of freedom relative to the position (e.g., x, y, z) and three angular degrees of freedom measuring the orientation of the tip of the medical instrument (e.g., yaw, pitch, roll). The data characterizing the pose may be relative to a coordinate system defined by the sensor system, as may be defined based on an EM field generator or a pose defined by a known position.

Various modules of the of the control circuitry 251 may process the data stored in the data store 260. For example, the control circuitry 251 may include a pose recommendation module 240, a navigation system 242, and an external imaging device controller 244. The pose recommendation module 240 may be control circuitry configured to generate a suggested pose (e.g., a position and/or angle) for an external imaging device, such as the external imaging device 70 shown in FIG. 1. The operations of the pose recommendation module 240 are discussed in greater detail below but may generally involve receiving data indicative of a location of a medical instrument and from that, generating an imaging angle for the external imaging device.

The navigation system 242 may include control circuitry that processes various data in the data store to generate navigation information to an operator of the system 100. In some embodiments, the navigation system 242 may provide pose information of the medical instrument relative to a three-dimensional model, an area of interest for a procedure, and the like. Further, the navigation system 242 may provide other information that may be helpful for a user of the system 100. As is explained in greater detail below, the navigation system 242 may provide suggested pose data for the external imaging device to an operator of the system 100 via the displays 56. This may be done so that the operator can cause the external imaging device to conform to the suggested pose data, especially on those embodiments where the control system 50 does not have direct control over the operation of the external imaging device 70.

The imaging device controller 244 may include control circuitry that provides feedback and/or control to the external imaging device 70. In some embodiments, the imaging device controller 244 may provide input commands to the imaging device 70 to cause the external imaging device 70 to adjust it's positioning to conform with a recommended imaging angle provided by the pose recommendation module 240.

### Three-Dimensional Model Reconstruction Methods and Operations

Details of the operations of exemplary pose recommendation systems are now discussed. The methods and operation disclosed herein are described relative to the pose recommendation system 100 shown in FIG. 1 and the modules and other components shown in FIGS. 2. However, it is to be appreciated that the methods and operations may be performed by any of the components, alone or in combination, discussed herein.

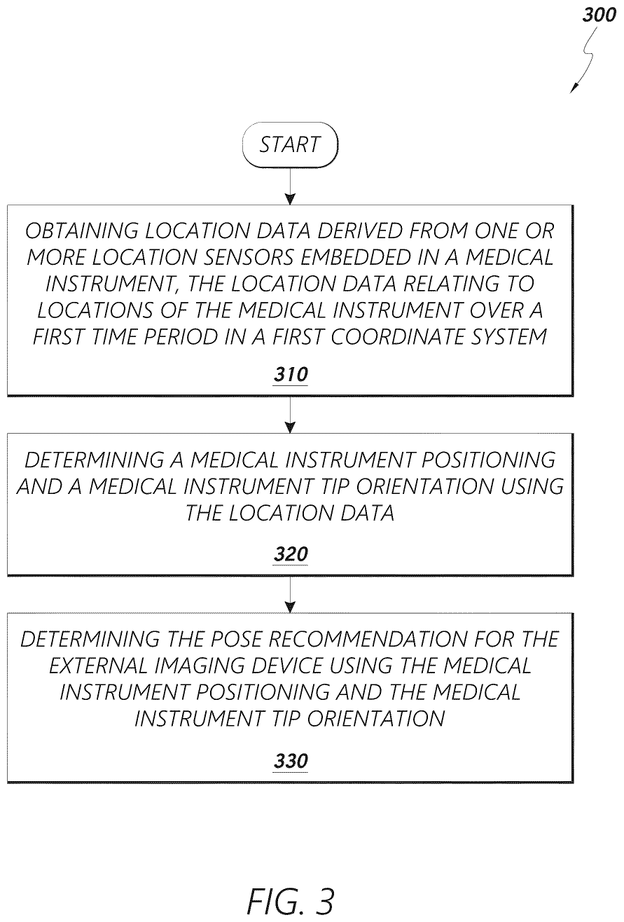

FIG. 3 is a flow-chart illustrating a method 300 for generating a pose recommendation for an external imaging device, according to an example embodiment. As FIG. 3 shows, the method 300 may begin at block 310, where the pose recommendation system 100 obtains location data derived from one or more location sensors embedded in a first medical instrument. As described above, a medical instrument may include a location sensor (e.g., robotized sensors, IMUs, fiber-optic shape sensors, EM sensors, and camera sensors) that generates sensor data. The sensor data generated by the location sensor may utilize a coordinate space different from the coordinate space used to control the external imaging device. For example, the location data of block 310 may, in some embodiments, be relative a coordinate frame used by a sensor system to which the location sensor belongs. In embodiments where the location sensor is an EM sensor, the location data may be expressed according to a coordinate frame defined by the EM field generator. As another example, in embodiments where the location sensor is a shape sensing fiber or an IMU-style location sensor, the location data may be expressed according to a coordinate frame relative to a known location.

Block 310 may involve transforming the location data relative to a coordinate frame that is different from the coordinate frame used by the location sensor. For example, some embodiments may transform the sensor data derived by the location sensor to a coordinate frame of the patient body. Some embodiments may be capable of performing a direct transformation from location sensor coordinate frame to the coordinate frame of the patient body based on a transform generated as part of a process that registers the location sensor to a patient body. Other embodiments may infer a mapping between patient coordinate space and location sensor coordinate space (and vice-versa) based on a registration between the coordinate frame of the location sensor and a coordinate frame of a virtual model of the patient's anatomy. This mapping may be performed by the navigation system 242. In embodiments that map the location sensor data to a coordinate frame of a virtual model, block 310 may involve the navigation system performing the transformation of the location data from the coordinate system of the sensor system to the coordinate system of the virtual model.

At block 320, based on the location data obtained at block 310, the system determines a medical instrument positioning and a medical instrument tip orientation. As discussed above with reference to FIG. 1, the robotic system 10 controls motion of the medical instrument 40 and can cause linear movement of the medical instrument based on linear movement of the end effector of the robotic arm controlling the medical instrument. Further, with continued reference to FIG. 1, the robotic system 10 may control the medical instrument 40 and cause articulation at the distal section of the medical instrument 40 to affect angular changes at the distal tip. The movement of the medical instrument (linear motion such as insertion/retraction and articulation) may be characterized according to the data stores shown in FIG. 2. Further, as discussed, this characterization may be according to any number coordinate systems, such as coordinate systems of: the sensor systems, anatomical models, the patient, the robot, the external imaging device, and the like.

Accordingly, some embodiments of block 320 of FIG. 3 involves using the location data, over a time period, to characterize the positioning in which the medical instrument has moved laterally within the anatomy and an orientation of the tip of the medical instrument within the anatomy. It is to be appreciated that “orientation” indicates angular information of the instrument tip, and may include overall roll, pitch, and yaw in relation to the anatomical model as well as pitch, roll, raw within an identified branch. This angular information may be derived from the location sensor of the medical instrument or determined from the robotic data and, if needed, transformed to a coordinate system consistent with the patient anatomy, such as via a transform to the virtual model of the anatomy, as discussed above.

At block 330, based on the medical instrument positioning and the medical instrument tip orientation (as determined at block 320), the system 100 determines a pose recommendation for the external imaging device. In some embodiments, block 330 involves the system 100 determining a plane formed by the medical instrument positioning and the medical instrument tip orientation and projecting a vector normal to that plane to represent an external imaging device angle that produces a visualization plane that contains the scope positioning and is constrained by the orientation of the medical instrument tip. This visualization plane will then include both the scope location and the orientation in which the medical instrument is pointing, as well providing the axis that the medical instrument (or working channel instrument) insertion happens.

### Medical Instrument Positioning and Tip Orientation

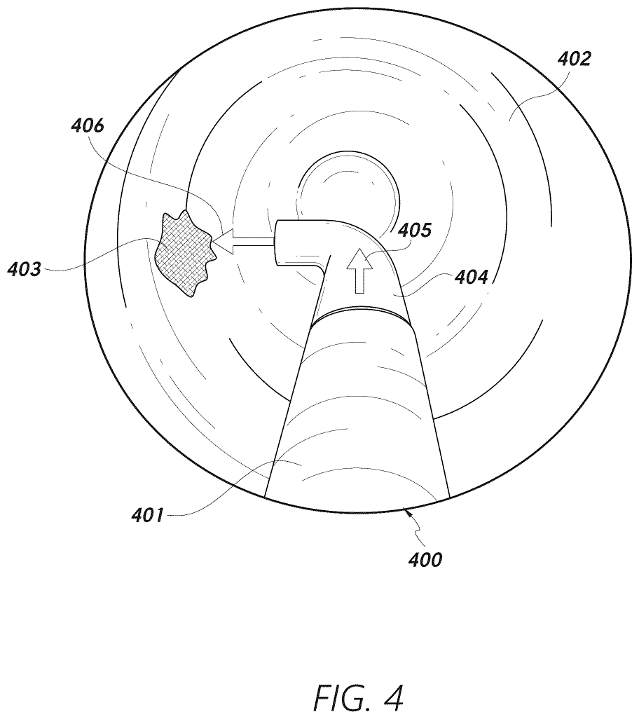

As discussed above with reference to blocks 320 and 330 of FIG. 3, the system 100 determines a pose recommendation for the external imaging device based on data indicative of a medical instrument positioning within a patient anatomy and a medical instrument tip orientation (e.g., angular information, such as yaw, pitch, roll) within a patient anatomy. The concepts involving the generation of the pose recommendation are now described in greater detail. FIG. 4 illustrates the distal end of a medical instrument within an anatomical lumen, in accordance with an exemplary embodiment. In FIG. 4, the medical instrument 400, comprising a shaft 401, is shown navigating through an anatomical lumen 402 towards an operative site 403 in positioning 405. In some embodiments, the system achieves movement of the medical instrument 400 in the positioning 405 based on an insertion movement of the distal end of the robotic arm controlling the medical instrument.

Having reached the operative site 403, the control system 10 may cause an articulation section 404 to bend in the orientation marked by arrow 406 in order to direct tools (e.g., working channel instruments such as, for example, needles, brushes, forceps, and the like) towards operative site 403. In some embodiments, the system achieves articulation of the medical instrument 400 towards the arrow 406 based on an actuation of motors in the end effector of the robotic arms that may, for example, cause tension on pull-wires extending through the medical instrument. This tension on the pull-wires may cause a bend in the articulation section 404.

The system 100 may collect location data regarding the movement of the medical instrument through the anatomical lumen 402. The system may use the location data to determine the medical instrument positioning 405 and the medical instrument orientation in relation to the arrow 406 shown in FIG. 4. For example, the location data may provide 6 DOF in sensor coordinate space, which can then be mapped to the patient coordinate space, either based on a direct registration to the patient coordinate space or based on a registration to a virtual model of an anatomy and a correspondence (implied or otherwise) between the coordinate spaces of the virtual model and the patient.

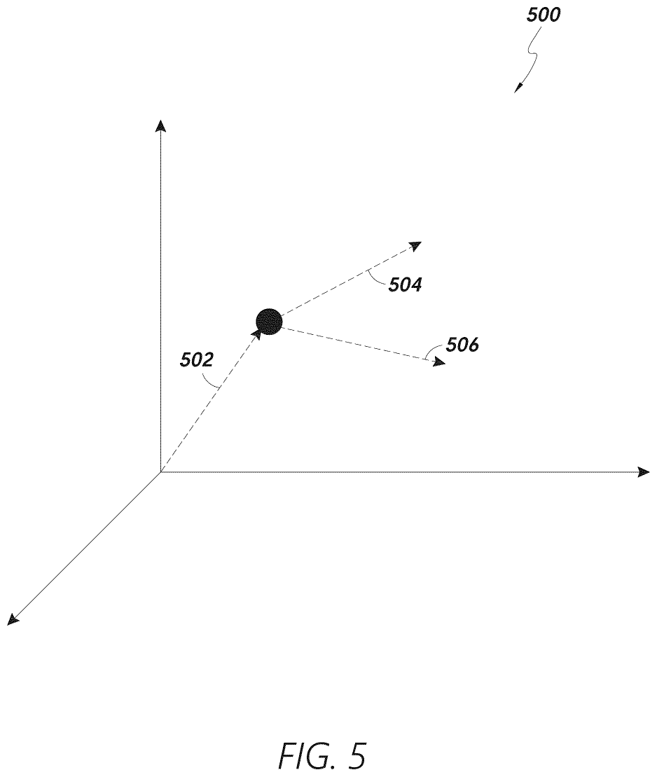

As described with reference to block 330 of FIG. 3, the system may, as part of determining a pose recommendation, determine an imaging vector relative to the medical instrument positioning and the medical instrument orientation. FIG. 5 is a diagram 500 illustrating an imaging vector 506 that is based on a medical instrument positioning 502 and a medical instrument orientation 504, according to an example embodiment. The medical instrument positioning 502 and the medical instrument orientation 504 are vectors representing location data acquired from one or more sensors associated with a medical instrument over a time period, consistent with the embodiments discussed herein. The imaging vector 506 is a vector representing a normal to a plane created by the medical instrument positioning 502 and the medical instrument orientation 504. As shown in FIG. 5, the imaging vector 506 may be determined based on a cross product of the medical instrument positioning 502 and the medical instrument orientation 504.

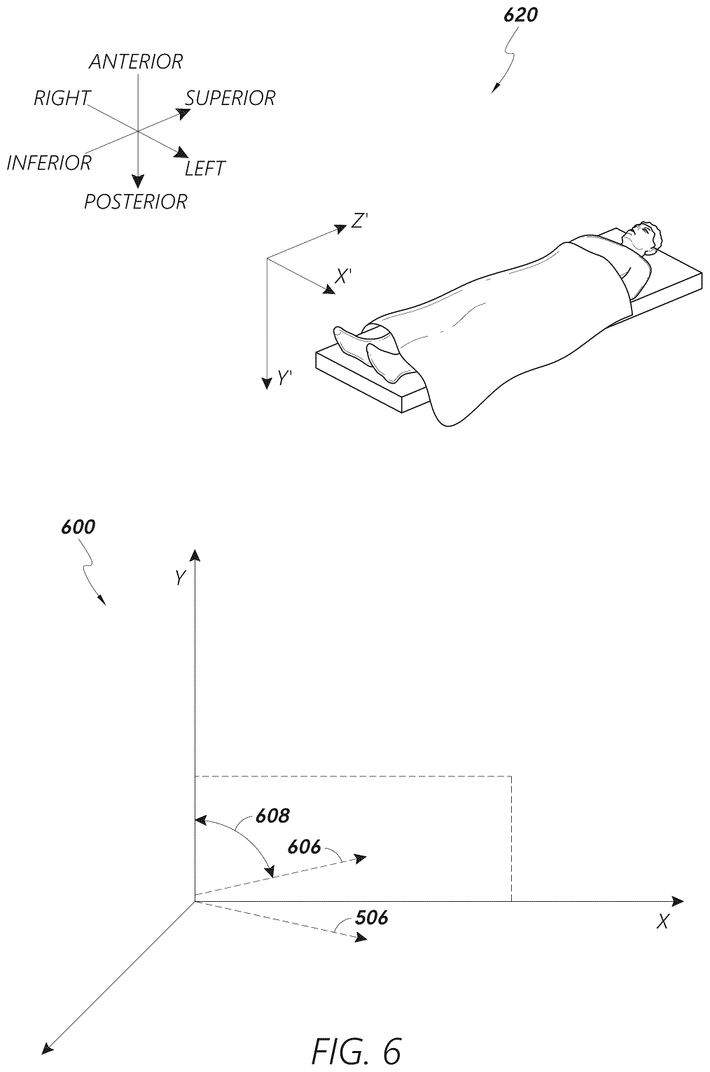

FIG. 6 is a diagram 600 showing the imaging vector 506 shown in FIG. 5 projected onto the X-Y plane of the external imaging device coordinate system, according to an example embodiment. FIG. 6 illustrates that the system 100 according to some embodiments will project the imaging vector 506 onto the X-Y plane of the imaging device coordinate system, thereby defining a projected imaging vector 606. In some embodiments, the gantry moves in a plane perpendicular to the patient body, which is the same as the X′-Y′ plane of the of the patient coordinate system, shown in callout 620. Since coordinate system of the medical instruments and external imaging device/patient coordinate systems can be registered together, the systems discussed herein can determine medical instrument tip position/orientation on CT/patient coordinate system based on medical instrument tip position/orientation in instrument coordinate system.

The angle 608 created from the projected imaging vector 606 and the Y axis defines a suggested angle for the external imaging device.

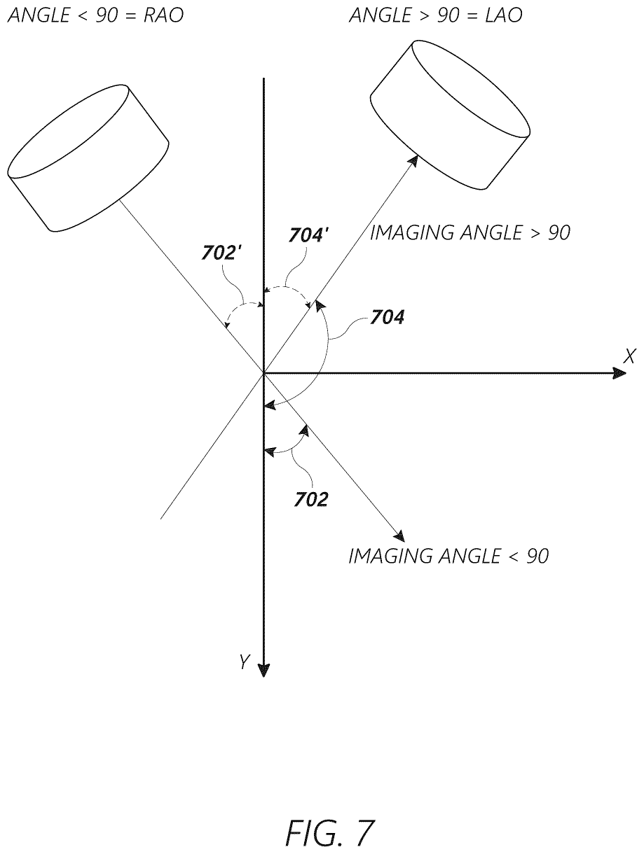

In some embodiments, a suggested angle (as may be surfaced to the operator of the system 100 or sent as a command to the external imaging device) may be based on a modification of the projected angle 608 to reflect an actionable movement of the external imaging device to achieve the desired imaging plane. FIG. 7 is a diagram illustrating left and right motions based on different values (e.g., angle 702 and angle 704) for the angles created from the projected imaging vector, according to an example embodiment. As shown in FIG. 7, for projected angle values that are less than 90 degrees, such as angle 702 which may be measured as A degrees, the system 100 may move the external imaging device A degrees (angle 702′) to the left to achieve a right anterior oblique view. Also, for angle values that are greater than 90 degrees, such as angle 704 which may be measured as B degrees, the system may move the external imaging device 180−B degrees (180 degrees minus B degrees; angle 704′) to the right to achieve a left anterior oblique view.

It is to be appreciated that by using the medical instrument positioning and the medical instrument orientation to generate an imaging vector, the visualization plane will contain the medical instrument positioning. Further, by constraining this visualization plane with the medical instrument orientation (e.g., yaw, pitch, and roll of the tip of the medical instrument), the image captured by the external imaging device will contain both the medical instrument location and the orientation to which the scope is pointing. This, in turn, may provide the axis that the tool insertion happens.

### Surfacing the Suggested Imaging Angle

As previously discussed, the suggested imaging angle for the external imaging device may be sent to the external imaging device through a communication interface in embodiments where the external imaging device is integrated with the control system 50. However, some embodiments may include a control system that lacks the capability for actuating control of the external imaging device. In either of these embodiments, the control system 50 may generate a notification of the suggested imaging angle to the operator. For example, some embodiments may cause a display device to render a user interface that displays the suggested imaging angle.

For embodiments that lack an integrated external imaging device, notifying an operator of the suggested imaging angle may provide actionable information to the operator of the system 100 so that they can then cause the external imaging device to move in a way to achieve the suggested imaging angle and, in turn, be in a position to acquire comparatively improved imaging of a surgical site.

Further, for embodiments that include an integrated external imaging device, surfacing the suggested imaging angle may provide the operator with information that they can confirm before proceeding or provide contextual information on the operation of the components of the system. For example, in embodiments where the control system controls movement of the external imaging device, a notification of the suggested imaging angle may allow the operator to trouble shoot whether the control system is moving the external imaging device to the suggested imaging angle, as intended.

FIG. 8 is a diagram of a user interface 800 that includes a suggested imaging angle 802, according to an example embodiment. The user interface 800 may be rendered by the control system 50 described above. By way of example and not limitation, the suggested imaging angle 802 may provide an angle and a direction relative to the patient coordinate space. The suggested imaging angle 802 may be determined by any of the components and methods described herein. As shown in FIG. 8, the suggested imaging angle 802 indicates that the external imaging device (e.g., a C-arm) is expected to move 20 degrees to the right.

It is to be appreciated that although the above discusses a graphical user interface for notifying the operator of the suggested imaging angle, other embodiments contemplated by this disclosure are not so limited. For example, some embodiments may use any other suitable notification including sound (e.g., voice), haptic feedback, other visual notification (e.g., a visual depiction of the angle), or any combination thereof.

### Implementing Systems and Terminology

Implementations disclosed herein provide systems, methods and apparatus to generate a pose recommendation for an external imaging device in a medical system. Various implementations described herein provide for improved visualization of an anatomy during a medical procedure using a medical robot.

The system 100 can include a variety of other components. For example, the system 100 can include one or more control electronics/circuitry, power sources, pneumatics, optical sources, actuators (e.g., motors to move the robotic arms), memory, and/or communication interfaces (e.g., to communicate with another device). In some embodiments, the memory can store computer-executable instructions that, when executed by the control circuitry, cause the control circuitry to perform any of the operations discussed herein. For example, the memory can store computer-executable instructions that, when executed by the control circuitry, cause the control circuitry to receive input and/or a control signal regarding manipulation of the robotic arms and, in response, control the robotic arms to be positioned in a particular arrangement.

The various components of the system 100 can be electrically and/or communicatively coupled using certain connectivity circuitry/devices/features, which can or may not be part of the control circuitry. For example, the connectivity feature(s) can include one or more printed circuit boards configured to facilitate mounting and/or interconnectivity of at least some of the various components/circuitry of the system 100. In some embodiments, two or more of the control circuitry, the data storage/memory, the communication interface, the power supply unit(s), and/or the input/output (I/O) component(s), can be electrically and/or communicatively coupled to each other.

The term “memory” is used herein according to its broad and ordinary meaning and can refer to any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

Computer-readable media that can be implemented in accordance with embodiments of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. may be either X, Y, or Z. Thus, such conjunctive language is not generally intended to imply that certain embodiments require at least one of X, at least one of Y, and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the disclosure should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method for generating a recommended pose for an imaging device external to a subject, the method comprising:
obtaining location data from one or more location sensors associated with a medical instrument internal to the subject,
determining, based on the location data, a change in position of the medical instrument over a period of time and an orientation of a tip of the medical instrument relative to a first coordinate frame; and
determining the recommended pose for the imaging device based on the change in position of the medical instrument and the orientation of the tip.

2. The method of claim 1, wherein the recommended pose includes an angle in which the imaging device is to be directed relative to a second coordinate frame.

3. The method of claim 1, further comprising generating a notification indicating the recommended pose.

4. The method of claim 3, wherein the notification includes a graphical representation of the recommended pose.

5. The method of claim 1, further comprising commanding the imaging device to move in accordance with the recommended pose.

6. The method of claim 1, wherein determining the recommended pose for the imaging device comprises determining an imaging plane formed by the change in position of the medical instrument and the orientation of the tip.

7. The method of claim 6, wherein the recommended pose is associated with a vector normal to the imaging plane.

8. The method of claim 7, wherein the determining of the recommended pose further comprises projecting the vector onto a second coordinate frame associated with the imaging device.

9. The method of claim 1, wherein the location data includes location sensor data, robotic data, image data, or any combination thereof.

10. The method of claim 1, further comprising adjusting the recommended pose based on a range of movement actionable by the imaging device.

11. A system for generating a recommended pose for an imaging device external to a subject, the system comprising:
a medical instrument;
one or more location sensors associated with the medical instrument;
control circuitry; and
a computer-readable medium, the computer-readable medium having instructions that, when executed, cause the control circuitry to:
obtain location data from the one or more location sensors while the medical instrument is inside the subject;
determine, based on the location data, a change in position of the medical instrument over a period of time and an orientation of a tip of the medical instrument relative to a first coordinate frame; and
determine the recommended pose for the imaging device based on the change in position of the medical instrument and the orientation of the tip.

12. The system of claim 11, wherein the orientation of the tip is associated with a second coordinate frame different than the first coordinate frame.

13. (canceled)

14. The system of claim 11, wherein the imaging device is configured to capture fluoroscopic images.

15. The system of claim 11, further comprising commanding the imaging device to move in accordance with the recommended pose.

16. The system of claim 11, wherein determining the recommended pose comprises:
determining an imaging plane using the medical instrument positioning and the medical instrument tip orientation;
determining a vector normal to the imaging plane; and
projecting the vector onto a second coordinate frame associated with the imaging device.

17. (canceled)

18. (canceled)

19. The system of claim 11, wherein the location data includes location sensor data, robotic data, image data, or any combination thereof.

20. The system of claim 11, further comprising adjusting the recommended pose based on a range of movement actionable by the imaging device.

21. A system for generating a recommended pose for a fluoroscopic imaging device, the system comprising:
a medical instrument having a distal tip;
one or more electromagnetic (EM) sensors associated with the medical instrument;
control circuitry; and
a computer-readable medium, the computer-readable medium having instructions that, when executed, cause the control circuitry to:
obtain location data from the one or more EM sensors while the medical instrument is inside a subject;
determine, based on the location data, a change in position of the medical instrument over a period of time relative to a coordinate frame associated with an EM field generator and an orientation of the distal tip representing one or more angular directions relative to a coordinate frame associated with an anatomy; and
determine a recommended pose for the fluoroscopic imaging device based on the change in position of the medical instrument and the orientation of the tip.

22. (canceled)

23. (canceled)

24. (canceled)

25. (canceled)

26. (canceled)

27. (canceled)

28. (canceled)

29. The system of claim 11, wherein the one or more location sensors include one or more electromagnetic (EM) sensors.

30. The system of claim 11, wherein the orientation of the tip of the medical instrument represents an angular direction of the tip.
